(19) United States
(12) Patent Application Publication
(10) Pub. No.: US 2006/0068452 A1
(43) Pub. Date: Mar. 30, 2006

(54) Differential protein expression patterns related to disease states

(75) Inventors: Ira L. Goldknopf, The Woodlands, TX (US); Essam A. Sheta, The Woodlands, TX (US); Melanie B. Black, The Woodlands, TX (US); Chris W. Wilson, Spring, TX (US); Helen R. Park, Huntsville, TX (US)

Correspondence Address: ELIZABETH R. HALL, 1722 MARYLAND STREET, HOUSTON, TX, 77006, US

(73) Assignee: Power3 Medical Products, Inc., The Woodlands, TX (US)

(21) Appl. No.: 11/172,219

(22) Filed: Jun. 29, 2005

Related U.S. Application Data
(60) Provisional application No. 60/614,315, filed on Sep. 29, 2004.

(51) Int. Cl. G01N 33/574; G06K 9/00

(52) U.S. Cl. 435/7.23; 382/128

(57) ABSTRACT

The present invention is a method for determining protein expression profiles in disease or an altered biological state. The method is based on the use of two-dimensional (2D) gel electrophoresis where the gel images are assigned two different colors. The gels are then compared by an overlay procedure that allows for identification and quantification of unique proteins by determining which colors are detected in the superimposed images and the density of those colors.

### CROSS REFERENCE TO RELATED APPLICATIONS

This application claims priority to U.S. Provisional Patent Application Ser. No. 60/614,315 filed Sep. 29, 2004 and entitled “Differential Protein Expression Patterns Related to Disease States” by inventors Ira L. Goldknopf, et al.

### BACKGROUND OF THE INVENTION

1. Field of the Invention

The invention relates to a method for determining biomarkers and protein expression patterns and using those biomarkers and protein expression patterns to define diseases or altered biological states. The method is based on use of proteomic analysis to identify biomarkers and protein expression patterns that define disease states or alterations in normal biological processes.

2. Description of the Related Art

Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in screening and diagnosis for clinicians. However, proteonomic testing for diagnostic purposes remains in its infancy.

Detection of abnormalities in the genome of an individual can reveal the risk or potential risk for individuals to develop a disease. The transition from risk to emergence of disease can be characterized as an expression of genomic abnormalities in the proteome. This transition from potential to actuality occurs when genetic abnormalities begin the process of cascading effects that can result in the deterioration of the health of the patient. Therefore, early detection of proteomic abnormalities at an early stage is desired in order to allow for detection of disease either before it is established or in its earliest stages where treatment may be effective.

Recent progress using a novel form of mass spectrometry called surface enhanced laser desorption and ionization time of flight (SELDI-TOF) for the testing of ovarian cancer has led to an increased interest in proteonomics as a diagnostic tool (Petrocoin, E. F. et al. 2002. Lancet 359:572-577). Further, proteomics has been applied to the study of breast cancer through use of 2D gel electrophoresis and image analysis to study the development and progression of breast carcinoma (Kuerer, H. M. et al. 2002. Cancer 95:2276-2282).

In the case of breast cancer, breast ductal fluid specimens were used to identify distinct protein expression patterns in bilateral matched pair ductal fluid samples of women with unilateral invasive breast carcinoma. This method of diagnosing and monitoring breast cancer was detailed in U.S. Pat. No. 6,855,554, where a side-by-side comparison, either visually or by image analysis, was used to determine differences in protein expression profiles between cancerous breasts and those free of cancer. U.S. Patent Application No. 2003/0236632 A1 discloses specific biomarkers for breast cancer detection that comprise human CRIP1 or HN1 sequences. In U.S. Pat. No. 6,670,141 B2, a method for diagnosing and monitoring malignant breast carcinoma is disclosed. The method relies on detection of a panel of biomarkers in saliva samples wherein the biomarkers include cancer antigen 15-3, tumor suppressor oncogene protein 53, oncogene c-erb-2, and combinations thereof.

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning. U.S. Pat. No. 6,326,209 B1 discloses measurement of total urinary 17 ketosteroid-sulfates as biomarkers of biological age. U.S. Pat. No. 6,693,177 B1 discloses a process for preparation of a single biomarker specific for O-acetylated sialic acid and useful for diagnosis and outcome monitoring in patients with lymphoblastic leukemia.

The importance of identifying specific biomarkers has led to a continuing need for new procedures that can identify unique protein expression patterns in disease states, particularly patterns that would remain undetected by using currently available methods of analysis. This type of protein expression pattern analysis will be useful for both detection of disease or altered biological states as well as diagnosis of disease states.

### SUMMARY OF THE INVENTION

The present invention is a method for determining a pattern of protein expression for a disease or an altered biological state. A method for determining an altered pattern of protein expression comprising: a) collecting a biological sample from an individual having an altered biological state; b) performing a two-dimensional (2D) electrophoretic separation of a plurality of proteins in the biological sample to produce a sample 2D gel pattern; c) coloring the sample 2D gel pattern a first color; d) superimposing the sample 2D gel pattern over a control 2D gel pattern colored a second color, wherein the control 2D gel pattern represents a standard protein expression pattern of a control sample collected from an individual free of the altered biological state; e) aligning a set of standard proteins in the sample 2D gel pattern and the control 2D gel pattern to form an aligned overlay; and f) conducting an image analysis of the aligned overlay to identify and quantify a set of protein variations in the sample 2D gel pattern that differ from the control 2D gel pattern, whereby the set of protein variations is indicative of the altered biological state.

Another embodiment of the invention is a method for screening for an altered biological state comprising: a) collecting a biological sample from a subject; b) performing a two-dimensional (2D) electrophoretic separation of a plurality of proteins in the biological sample to produce a sample 2D gel pattern; c) coloring the sample 2D gel pattern a first color; d) superimposing the sample 2D gel pattern over a control 2D gel pattern colored a second color, wherein the control 2D gel pattern represents a standard protein expression pattern of a control sample collected from an individual free of the altered biological state; e) aligning a standard protein in the sample 2D gel pattern with the standard protein in the control 2D gel pattern to form an aligned overlay; and f) conducting an image analysis of the overlay to identify and quantify a set of protein variations in the sample 2D gel pattern that differ from the control 2D gel pattern, whereby the set of protein variations is indicative of the altered biological state.

A further embodiment of the invention is a method for determining a pattern of protein expression for an altered biological state comprising: a) collecting a first biological sample known to exhibit the altered biological state; b) collecting a second biological sample known not to exhibit the altered biological state; c) precipitating a first protein fraction from the first sample and a second protein fraction from the second sample; d) performing a two-dimensional gel electrophoretic analysis of the first protein fraction to produce a first 2D gel pattern; e) performing a two-dimensional gel electrophoretic analysis of the second protein fraction to produce a second 2D gel pattern; f) staining the first 2D gel pattern a first color and the second 2D gel pattern a second color; g) superimposing the first 2D gel pattern over the second 2D gel pattern to form an overlay; h) aligning the first 2D gel pattern with the second 2D gel pattern to maximize the presence of a third color in the overlay, wherein the third color results from mixing the first color and the second color; i) conducting an image analysis of the aligned overlay to identify a set of differentially expressed proteins in the first sample, whereby the set of differentially expressed proteins is indicative of the altered biological state.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a more complete understanding of the present invention, and the advantages thereof, reference is now made to the following descriptions taken in conjunction with the accompanying drawings, in which:

FIG. 1 illustrates the results of 2D gel image analysis of nipple aspirate fluid samples of the right and left breast of a woman that is free of breast cancer (normal woman). Panel A is the red stained image of the left breast sample while panel B is the green stained image of the right breast sample. Panel C shows the results of overlaying panel A and panel B.

FIG. 2 illustrates the results of 2D gel image analysis of nipple aspirate fluid samples of the right and left breast of a woman that has been diagnosed with unilateral breast cancer (patient). Panel A is the red stained image of the left breast sample while panel B is the green stained image of the right breast sample. Panel C shows the results of overlaying panel A and panel B.

FIGS. 3A and 3B illustrate results of 2D gel image analysis of NAF samples of the left (FIG. 3A) and right (FIG. 3B) breasts of a woman with a familial history of breast cancer, where the woman is currently mammogram negative for breast cancer.

FIG. 4 illustrates the results of a 2D gel image analysis of a nipple aspirate fluid sample of a normal breast with the three acetyl-LDL receptor spots marked.

FIG. 5 is a graph indicating the acetyl-LDL receptor concentrations of each breast of four normal women (N1-N4), two high-risk women (S1-S2), and twelve women diagnosed with unilateral breast cancer (P1-P12).

### DESCRIPTION OF THE PREFERRED EMBODIMENTS

The present invention is a sensitive method for determination of protein biomarkers and protein expression profile differences among biological samples taken from patients with and without disease or altered biological states.

In the context of the present invention a “disease” or “disease state” is a condition wherein an individual or patient exhibits a known set of symptoms or biological changes and would include but not be limited to cancer (e.g., breast cancer, prostate cancer, brain cancer, uterine cancer, ovarian cancer, ovarian cancer, leukemias, and lymphomas), neurodegenerative disease (e.g., Alzheimer's disease, ALS, Parkinson's disease, muscular dystrophy, and multiple sclerosis), and autoimmune diseases (e.g., SLE, etc.). An “altered biological state” is any situation where the individual's or patient's normal biological function has been shown to be different as compared to the function that individual had known previously, or what has been identified as normal in a population of individuals, wherein an altered biological state may include a disease state.

In the context of the present invention, the “protein expression profile” corresponds to the steady state level of the various proteins in the biological samples that can be expressed qualitatively or quantitatively. These steady state levels are the result of the combination of all the factors that control protein concentration in a biological sample. These factors include but are not limited to: the rates of transcription of the genes encoding the hnRNAs; the rates of processing of the hnRNAs into mRNAs; the splicing variations during the processing of the hnRNAs into mRNAs which govern the relative amounts of the protein isoforms; the rates of processing of the various mRNAs by 3′-polyadenylation and 5′-capping; the rates of transport of the mRNAs to the sites of protein synthesis; the rate of translation of the mRNA's into the corresponding proteins; the rates of protein post-translational modifications, including but not limited to phosphorylation, nitrosylation, methylation, acetylation, glycosylation, poly-ADP-ribosylation, ubiquitinylation, and conjugation with ubiquitin like proteins; the rates of protein turnover via the ubiquitin-proteosome system; the rates of intracellular transport of the proteins among compartments such as but not limited to the nucleus, the lysosomes, golgi, the membrane, and the mitochondrion; the rates of secretion of the proteins into the interstitial space; the rates of secretion related to protein processing; and the stability and rates of processing and degradation of the proteins in the biological sample before and after the sample is taken from the patient.

In the context of the present invention, the “disease protein footprint” of a particular disease or altered biological state is the differential protein expression profile between a normal or control sample and a sample expressing that particular disease or altered biological state. The word “footprint” is used in this context to describe the characteristics of the protein expression pattern that are indicative or define a particular condition. Much like a footprint of an animal in the forest can be studied to identify the type of animal, the protein disease footprint can be used to determine which disease processes are at work in the disease state of an individual patient. A specific protein footprint may be determined for control or normal samples and for a particular disease or altered biological state.

The biological samples employed with the present invention are samples from individuals suspected of having an altered biological state or disease (unknown samples) or control samples. A “control” sample is a sample from an individual known not to have the altered biological state or to be disease-free, or the “control” sample is one from the same individual but representative of cells or tissues not affected by the altered biological state or disease. An example of such a control sample would be the nipple aspirate fluid sample from a breast that is known to be non-cancerous and comparing it with the unknown sample of the nipple aspirate fluid from a breast suspected of being cancerous.

The method is based on the use of an overlay procedure, where an “overlay” is defined as the process of physically superimposing one stained 2D gel electrophoretic image over another stained 2D gel electrophoretic image. The two 2D gels are preferably stained with the same stain; however, they may be stained with two different stains as long as the stain intensity is standardized.

In order to detect the protein expression differences between the two images, each of the 2D gel images are assigned a different color. Preferably, the stains or dyes employed will react with the protein in the gel to provide an intensity of color that is proportional to the quantity of the protein present. The intensity of resulting color pattern is digitized and a color assigned to each stained image. The colors assigned to the first stained image (i.e., the first color) and the second stained image (i.e., the second color) are typically at different ends of the color spectrum so that if an equal intensity of the colors are added together one would get a third color (an additive color).

For example, if the first and second stained images were from the same biological sample, the two stained images would be substantially similar. If the first stained image is assigned red and the second stained image is assigned green, the overlay of the red over the green would yield an overlay predominately exhibiting the additive color yellow. In contrast, if the two stained images were similar samples (i.e., serum samples) collected from two different individuals, one individual having a disease and the other individual known to be free of the disease, then an overlay of the two stained images would be yellow where constitutively expressed proteins were present and a variety of colors where differentially expressed proteins were present. The protein expression profile difference between the two overlaid images is detected by visual inspection and/or image analysis.

Previous methods have used a side-by-side comparison of images only. In contrast, the present method uses a visual inspection and/or computerized image analysis of the overlay of two 2D gel electrophoretic images to determine quantitative and qualitative changes between the samples being compared.

Sample Collection and Preparation

In certain embodiments the biological samples may be subjected to pre-fractionation protocols such as preparative isoelectric focusing using any one of a number of devices such as a Rotofor (Bio-Rad Laboratories) and commercially available ampholytes, and/or subjected to precipitation by any number of reagents alone or in combination such as ammonium sulfate, trichloroacetic acid, perchloric acid, acetone, ethanol, commercial precipitant cocktails such as PlusOne (Amersham Biosciences), or Perfect-Focus (Geno Technology Inc.). Additionally, such samples may be subjected to any of immunoprecipitants, affinity capture using solid phase media such as anti-phosphotyrosine antibodies, other antibodies, lectins, attached to chromatography media such as agarose, or any other method designed to separate proteins from a solution into groups or from contaminants such as lipids, nucleic acids, carbohydrates, salts, or other substances not required for or interfering with testing.

Sample collection and storage may be performed in many different ways depending on the type of sample and the conditions of the collection process. One of skill in the art would apply sample collection techniques well known in the art. In one embodiment, nipple aspirate fluid was the sample type collected. The nipple aspirate samples were collected using a simple, non-invasive, suction device similar to a manual breast pump. The samples were collected, diluted with cold buffer (e.g., isotonic saline, Tris HCl, RPMI and the like) containing a mixture of protease inhibitors (e.g., PMSF, leupeptin, pepstatin, chymostatin, calpain inhibitor I, calpain inhibitor II, EDTA-free protease inhibitor cocktail, and the like) and frozen at 0° C. or below.

Two Dimensional-Electrophoresis of Samples

The protein profiles of the present invention are obtained by subjecting biological samples to two-dimensional (2D) gel electrophoresis to separate the proteins in the biological sample into a two-dimensional array of protein spots. In the context of the present invention a “biological sample” can be any sample obtained from the body of a patient including but not limited to whole blood, plasma, serum, urine, vaginal fluid, seminal fluid, cerebrospinal fluid, nipple aspirate fluid, vitreous fluid, bile, or an extract of any tissue of the body.

Two-dimensional gel electrophoresis is a useful technique for separating complex mixtures of proteins and can be performed using a variety of methods known in the art (see, e.g., U.S. Pat. Nos. 5,534,121, 6,398,933 and 6,855,554).

In certain embodiments, the first dimensional gel is an isoelectric focusing gel and the second gel is a denaturing polyacrylamide gradient gel. In certain embodiments, the sample may also be subjected to other various techniques known for separating proteins, techniques that would include but not be limited to gel filtration chromatography, ion exchange chromatography, reverse phase chromatography, affinity chromatography (typically in an HPLC or FPLC apparatus), or any of the various centrifugation techniques well known in the art. In some cases, a combination of one or more chromatography or centrifugation steps may be combined via electrospray or nanospray with mass spectroscopy or tandem mass spectroscopy, or any protein separation technique that determines the pattern of proteins in a mixture either as a one-dimensional, two-dimensional, three-dimensional or multi-dimensional pattern or list of proteins present.

Proteins are amphoteric, containing both positive and negative charges and like all ampholytes exhibit the property that their charge depends on pH. At low pH, proteins are positively charged while at high pH they are negatively charged. For every protein there is a pH at which that protein is uncharged (i.e., the isoelectric point or pI). When a charged molecule is placed in an electric field it will migrate towards the opposite charge. In a pH gradient such as those used in the present invention, a protein will migrate to the point at which it reaches its isoelectric point and becomes uncharged. The uncharged protein will not migrate further and stops. Each protein will stop at its isoelectric point and the proteins can thus be separated according to charge. In order to achieve optimal separation of proteins, various pH gradients may be used. For example, a very broad range of pH, from about 3 to 11 or 3 to 10 can be used, or a more narrow range, such as from pH 4 to 7 or 7 to 10 or 6 to 11 can be used. The choice of pH range is determined empirically and such determinations are within the skill of the ordinary practitioner and can be accomplished without undue experimentation.

In the second dimension, proteins are typically separated according to molecular weight by measuring mobility through a polyacrylamide gradient in the detergent sodium dodecyl sulfate (SDS). In the presence of SDS and a reducing agent such as dithiothreitol (DTT), the proteins act as though they are of uniform shape with the same charge to mass ratio. The proteins are then separated by molecular weight on the gel. It is well known in the art that various concentration gradients of acrylamide may be used for such protein separations. For example, a gradient of from about 5% to 20% may be used in certain embodiments or any other gradient that achieves a satisfactory separation of proteins in the sample may be used. Other gradients would include but not be limited to from about 5 to 18%, 6 to 20%, 8 to 20%, 8 to 18%, 8 to 16%, 10 to 16%, or any range as determined by one of skill.

Reproducibility of the Image Analysis

To assess the reproducibility of the 2D gel system, 75 ng of bovine serum albumin (BSA) was run on 9 separate 2D gels. The gels were stained with SYPRO RUBY (Bio-Rad Laboratories) and the 5 spots that resulted in the BSA region of the gel were then subjected to quantitative analysis using PDQUEST (Bio-Rad Laboratories) and the Gaussian Peak Value method. The results shown in Table 1 show that the electrophoretic patterns were reproducible and independent of the spot amount over the range tested.

TABLE 1
Reproducibility of Quantitation in 2D Gels - PDQuest
Peak Value of the Major Components of BSA

| | Spot # | | | | |
|---|---|---|---|---|---|
| Replicate # | 9901 | 9902 | 9904 | 9905 | 9906 |
| 1 | 332 | 1152 | 2612 | 739 | 229 |
| 2 | 246 | 974 | 2694 | 513 | 167 |
| 3 | 336 | 1065 | 2354 | 668 | 225 |
| 4 | 311 | 1272 | 3482 | 713 | 198 |
| 5 | 351 | 1168 | 2724 | 733 | 245 |
| 6 | 268 | 1059 | 2753 | 622 | 184 |
| 7 | 452 | 1630 | 4000 | 946 | 281 |
| 8 | 405 | 1195 | 2752 | 870 | 274 |
| 9 | 258 | 1050 | 2716 | 699 | 189 |
| Avg | 329 | 1174 | 2899 | 723 | 221 |
| Stdev | 68 | 193 | 510 | 127 | 40 |
| CV | 21% | 16% | 18% | 18% | 18% |
| ng/spot | 4.4 | 15.6 | 38.6 | 9.6 | 2.9 |

Quantitation of Proteins

In order to quantitate the amount of a particular stained protein detected in different samples, a standard curve for that particular protein was used. Typically, increasing amounts of a selected protein were added to a sample, separated by electrophoresis and stained. The density of stain at each known protein concentration was determined by image analysis and a standard curve prepared. In this way, the stain density can be linked to a particular protein concentration on the standard curve.

Comparison of the stain density for that protein in an unknown sample or a sample from a patient to the standard curve will allow for quantitation of the amount of protein present in the sample. Alternatively, the amounts of protein detected can be determined in different samples in comparison to a normal sample from a population. In the context of the present invention a “normal” sample is one wherein the sample has been determined to be representative of individuals without the disease or altered biological state being investigated. The normal sample is assigned a value of 100% and then each patient or unknown sample is compared to the normal sample's stain density.

Image Analysis

Gel images are compared visually and/or electronically. The gels are stained with a dye, including but not limited to Comassie blue, silver staining, and SYPRO RUBY. Typically, a SYPRO RUBY fluorescent stain is the dye of choice as it is a very sensitive dye that stains proteins in a quantitative manner.

Placing the stained SDS PAGE gels on the imaging platform of a FX-PRO Laser Scanner and scanning an image of the stained gel into the PDQUEST software program initiates one embodiment of the image analysis procedure of the present invention. The software is set for acquisition by selecting the Protein Stained Gel-SYPRO RUBY-High Intensity application, selecting the scan area to encompass the gel region on the platform, and selecting the resolution to 100 micrometers. By selecting the “acquire” button on the screen, the software performs the scanning operation. The resulting gel image is then ready for image analysis.

The process of image analysis for the gels begins by cropping the images to be analyzed and filtering them to eliminate the stain precipitate. The cropping must be done such that the protein patterns can be compared using the Multichannel viewer option in PDQUEST. This is generally accomplished by rotating the image and/or adjusting the cropped image horizontally or vertically. The images to be compared must be the same size as measured in pixels. The PDQUEST software has an image option that allows the user to reduce or expand the file size without distorting the image.

Two stained gel images are selected for comparison of their protein expression patterns and the protein pattern of each image is assigned a different color. The Multichannel viewer produces gel images with black backgrounds and colored protein patterns. The colors assigned to the first stained image (i.e., the first color) and the second stained image (i.e., the second color) are typically at different ends of the color spectrum so that if an equal intensity of the colors are added together one would get a third color (an additive color). The two stained images are then overlaid to align their protein expression patterns as closely as possible.

The protein expression profile of two 2D gels images are compared by scanning each of the two gels to be compared, marking the locations and X, Y coordinates of known proteins (i.e., standards) in both gels, and performing a match of the marked protein spots to provide specific reference points between the two gels. Once the protein standards in the two gels are aligned, the two protein scans are analyzed by the software and all of the spots with different X,Y coordinates are reported. Unfortunately, this process vastly overestimates the number of differentially expressed proteins.

Preferably, the two 2D gel images to be compared are each stained or assigned a distinct color, preferably at the opposite end of the color spectra from each other, and the two colored images are overlaid, either physically or electronically. The resulting color of each of the protein spots is quite informative. If a specifically identified spot in one gel is overlaid with the synonymous spot of the second gel to give the additive color, then the protein represented by those synonymous protein spots is made in similar quantities in each of the two samples being analyzed and is probably a constitutively expressed protein.

On the other hand, whenever a specifically identified spot in one gel is overlaid with the synonymous spot in the second gel to yield a non-additive color closer to the spectra of the first or second color, then the protein represented by those synonymous protein spots is made in different quantities in each of the two samples being analyzed and is a differentially expressed protein. If the resulting color of the overlaid spots is closer to the wavelength of the color assigned to the first gel, the concentration of that specific protein in the second gel is lower than in the first gel. Whereas, if the resulting color of the overlaid spots is closer to the wavelength of the color assigned to the second gel, the concentration of that specific protein in the second gel is greater than in the first gel. For example if the first gel is a protein expression pattern of a normal or control sample and the second gel is a protein expression pattern of an unknown the resulting color in the overlay of spots will indicate if that protein is differentially expressed in the unknown sample. Thus, if the resulting color of the overlaid spots is closer to the color of the normal or control sample then that protein is down-regulated in the unknown sample, and if the resulting color of the overlaid spots is closer to the color of the unknown sample then that protein is up-regulated in the unknown sample.

Since overlaying two distinctly different colored 2D gel images result in visually apparent color variations in the overlaid images, slight corrections in alignment patterns are readily made. In fact, the manual alignment of the two gel images to maximize the amount of the additive color seen in the overlaid gel images is very effective. Alternatively, one can select to have the 2D gel images electronically aligned to optimize the additive color.

Identifying a Protein Expression Profile Indicative of a Disease or an Altered Biological State

One of the significant advantages of overlaying two distinctly different colored gel images is that the predominate color variations in the overlaid images are visually apparent. As described above, whenever the two images are overlaid the portion of the protein expression patterns that are substantially identical appear as the additive color and the non-identical portions of the protein expression patterns appear as the first color, the second color or some color of an intermediate wavelength between the first and second color.

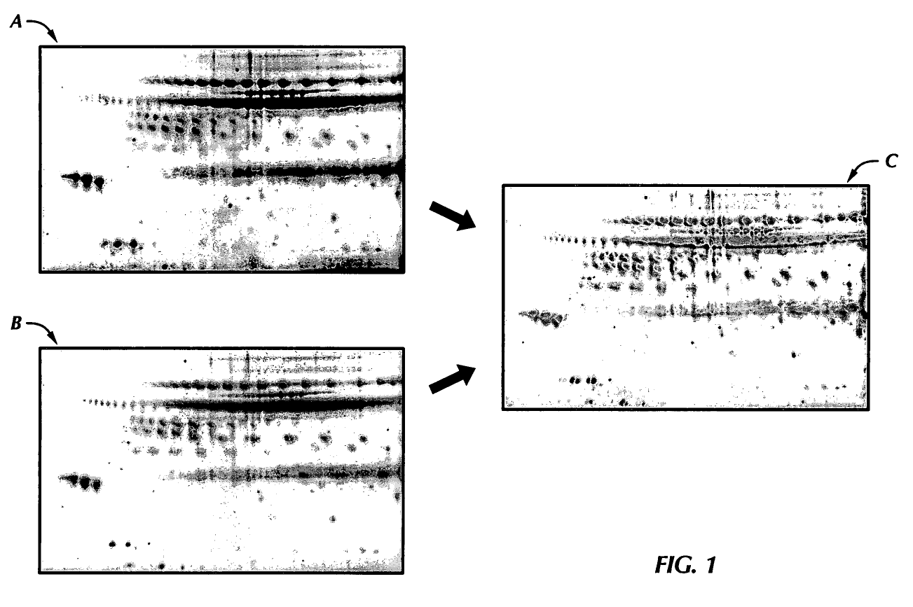

For example, if the first and second stained images were from the same biological sample, the two stained images would be substantially the same. If the first stained image is assigned red and the second stained image is assigned green, the overlay of the red over the green would yield an overlay predominately exhibiting the additive color yellow. Similarly, the predominate color of the overlay of a stained normal and a stained normal control image is the additive color. Thus, an unknown stained sample image overlaid with a stained normal control sample image will predominantly yield the additive color only when the unknown is a normal (as in FIG. 1).

Typically, as a number of protein expression patterns are obtained for normal or control samples and compared, a recognizable pattern or “normal protein footprint” becomes apparent among the control or normal samples and is highlighted in the overlaid images of normal or control samples by the consistent predominant appearance of the additive color in these overlaid control or normal samples.

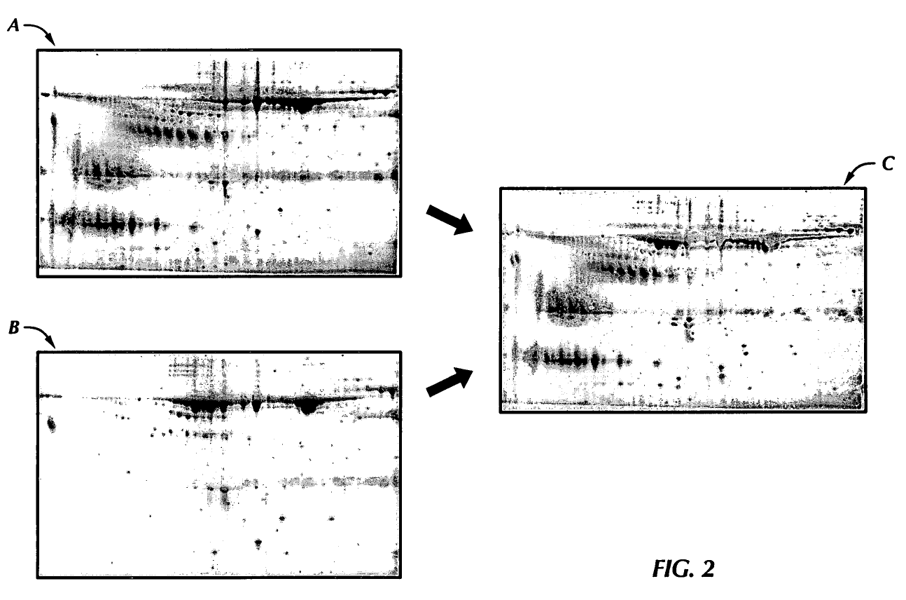

In contrast, if the two stained images were similar samples (e.g., nipple aspirate fluid samples) collected from two different individuals, one individual having a disease or an altered biological state and the other individual known to be free of the disease or altered biological state, then an overlay of the two stained images would be yellow where constitutively expressed proteins were present and a variety of colors where differentially expressed proteins were present. Thus, where the predominate color of the overlay of an unknown and the control gel image predominantly yields colors other than the additive color, the unknown contains a significant number of differentially expressed proteins and the unknown is designated a diseased or biologically altered state sample (see FIG. 2).

Although there are usually a number of distinctions among different individual's responses to an altered biological state or disease, often there are also commonalities among different individual's responses to a particular disease or altered biological state. As a number of protein expression patterns are obtained from samples collected from individuals known to have a specific disease or altered biological state and compared to normal or control samples, commonalities in the protein expression patterns of the disease or altered biological state become apparent as the “disease protein footprint.”

The evaluation of an unknown sample by the method of the present invention can be done visually, especially when the “control protein footprint” has only minor variations among control samples and the diseased samples contain several major differentially expressed proteins. Alternatively, especially where the control protein footprint or the disease protein footprint contains multiple variations, the analysis of the gel overlay may also be performed electronically. The gel overlay may be scanned at three wavelengths (i.e., the wavelengths of the first color, the second color, and the additive color). By plotting the three-wavelength scans of a number of gel overlays of normal samples overlaid with control samples, the control protein footprint can be determined. Similarly, by plotting the three-wavelength scans of a number of gel overlays of disease samples overlaid with the control protein footprint, the disease protein footprint can be determined.

Alternatively, a simple indication of whether an unknown sample is normal or not can be determined by performing an additive color wavelength scan of a gel overlay of the unknown sample and the control protein footprint and statistically comparing the total quantity of the additive color in the gel overlay to the total additive color seen in overlays of two control or normal samples.

The Isolation and Identification of Biomarkers for a Disease or an Altered Biological State

Furthermore, the quantity of a particular protein in two samples is identifiable as unchanged, absent, down-regulated, or up-regulated by the predominant color of that protein's spot in the gel image overlay of the two samples. The identification of particular proteins that are differentially expressed in the disease or altered biological state versus normal or control samples can then be identified and used as a biomarker of that disease or altered biological state. The selected protein spots are excised, in-gel digested with a protease, subjected to mass fingerprinting analysis by matrix-assisted laser desorption ionization-time of flight mass spectrometry (MALDI-TOF MS) and expert database searching.

Mass spectrometry provides a powerful means of determining the structure and identity of complex organic molecules, including proteins and peptides. The unknown compound is bombarded with high-energy electrons causing it to fragment in a characteristic manner. The fragments, which are of varying weight and charge, are then passed through a magnetic field and separated according to their mass/charge ratios. The resulting characteristic fragmentation pattern of the unknown compound is used to identify and quantitate the unknown compound.

MALDI-TOF MS is a type of mass spectrometry in which the analyte substance is distributed in a matrix before laser desorption. The analyte, co-crystallized with a matrix compound, is subjected to pulse UV laser radiation. The matrix, by strongly absorbing the laser light energy, indirectly causes the analyte to vaporize. The matrix also serves as a proton donor and receptor, acting to ionize the analyte in both positive and negative ionization modes. A protein can often be unambiguously identified by a MALDI-TOF MS analysis of its constituent peptides (produced by either chemical or enzymatic treatment of the sample).

Washing the gel spots with buffer and then soaking the gel spots in an organinc solvent, such as 100% acetonitrile, for at least 10 minutes destains the excised gel spots. After the gel spots are destained, the protein in the gel spot is digested with a protease, preferably trypsin.

Typically a small volume of trypsin solution (approximately 5-15 μg/ml trypsin) is added to the destained gel spots and incubated for 3 hours at 37° C. or overnight at 30° C. The digested peptides are extracted, washed, desalted and concentrated before spotting the peptide samples onto the MALDI-TOF MS target.

Mass spectral analyses of the digested peptides are performed to identify the selected protein. Those of skill in the art are familiar with mass spectral analysis of digested peptides. The mass spectral analysis was conducted on a MALDI-TOF Voyager DE STR (Applied Biosystems). Spectra were carefully scrutinized for acceptable signal-to-noise ratio (S/N) to eliminate spurious artifact peaks from the peptide molecular weight lists. Both internal and external standards were employed to calibrate any shift in mass values during mass spectroscopic analysis.

The external standards were a set of proteins having known molecular weights and known mass/charge ratios in their mass spectrum. A mixture of external standards is placed on the mass spectrophotometer chip well next to the well that includes an unknown sample. Internal standards are characteristic peaks in the sample spectrum that belong to peptides of the proteolytic enzyme (e.g., trypsin) used to digest the protein spots and extracted along with the digested peptides. Those peaks are used for internal calibration of any deviation of the spectral peaks of the sample.

Corrected molecular weight lists are then subjected to public database searches, such as the GenBank and dbEST databases maintained by the National Center for Biotechnology Information (hereinafter referred to as the NCBI database) and the SwissProt or Swiss Protein database maintained by ExPasy. Those of skill in the art are familiar with searching databases like the NCBI and SwissProt databases.

### EXAMPLE 1

### Differential Protein Expression Patterns in Breast Cancer

Breast ductal fluid samples were collected by nipple aspiration. The nipple aspirate fluid (NAF) samples were taken from 12 unilateral breast cancer patients, 4 normal women, and two mammogram negative women with a history of breast cancer in their family and where onset of disease had begun at the same age as their age when the samples were taken.

Each sample was first diluted with the addition of cold RPMI buffer containing an EDTA-free protease inhibitor cocktail. The diluted nipple aspirate fluid was aliquoted into 1.5 ml microfuge tubes in 100 μl portions and frozen in liquid nitrogen before analysis.

NAF samples were prepared for protein analysis by first washing with trichloroacetic acid (TCA) followed by two washes with acetone. This washing allowed for greater sensitivity of protein separation in the nipple aspirate fluid as compared to previous sample preparation methods, with more than 1200 proteins detected. In a preferred embodiment of the invention, NAF samples containing the protease inhibitor cocktail are taken from −80° C. and placed on ice for thawing. To each 100 μl of sample, 100 μL of LB-1 buffer (7M urea, 2M Thiourea, 1% DTT, 1% Triton X-100, 1× Protease inhibitors, and 0.5% Ampholyte pH 3-10) was added and the mixture vortexed. The sample was incubated at room temperature for about 5 minutes.

Then 300 μl UPPA-I (Perfect Focus, Genotech) was added to each tube, vortexed and incubated on ice for 15 minutes. Next 600 μl UPPA-II (Perfect Focus, Genotech) was added to each tube, vortexed and centrifuged at about 15,000×g for 5 minutes at 4° C. The entire supernatant was carefully removed by vacuum aspiration. Repeat centrifugation at about 15,000×g for 30 seconds was performed. The remaining supernatant was removed by vacuum aspiration.

The pellet was suspended in 25 μl of ultra pure H₂O and vortexed. Then 1 ml of OrgoSol (Perfect Focus, Genotech, prechilled at −20° C.) and 5 μl SEED (Perfect Focus, Genotech) were added to each pellet and incubated at −20° C. for about 30 minutes. The pellet was suspended using repeated vortexing bursts of about 20-30 seconds each. The tubes were then centrifuged at about 15,000×g for 5 minutes. The entire supernatant was carefully removed by vacuum aspiration. The water suspension and the OrgoSol-SEED wash of the pellet were repeated.

The protein pellet was air dried for about 5 minutes, then the pellet was dissolved in an appropriate amount of isoelectric focusing (IEF) loading buffer, incubated at room temperature and vortexed periodically until the pellet was dissolved to visual clarity. The samples were centrifuged briefly before a protein assay was performed on the sample.

An aliquot of 100 μg of NAF proteins was suspended in a total volume of 184 μl of IEF loading buffer and 1 μl Bromophenol Blue. Each sample was loaded onto an 11 cm IEF strip (Bio-Rad), pH 4-7, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 min with an equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 min incubation of the IEF strips in the equilibration buffer is performed as before, except freshly added iodoacetamide (C₂H₄INO) was added to a final concentration of 40 mg/ml. The IEF strips were then removed from the tray using clean forceps and washed five times in a graduated cylinder containing the Bio Rad running buffer 1× Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After running, the gels were carefully removed and placed in a clean tray and washed twice for 20 minutes in 100 ml of a pre-staining solution containing 10% methanol and 7% acetic acid.

Once the 2D gel patterns for the 16 women were obtained, the gels were stained with 100 ml of SYPRO RUBY fluorescent stain (Bio-Rad Laboratories) for 3 hours. The gels were destained for at least for 30 min before scanning.

The 2D gels were then scanned using a FX PRO laser scanner (Bio Rad) and the scanned images were analyzed by PDQUEST software imager (Bio-Rad Laboratories) as described above. Alternatively, the scanned gel images were converted to *.tiff files using the Bio-Rad PDQUEST software.

A user database was created in the Microsoft SQL server and activated. PROTEOMEWEAVER (Deifiniens AG) cognitive 2D analysis software was then activated and the *.tiff files were located and moved into the new experiment window of that software. The new experiment was named to reflect the two images to be overlaid, and the Match Matrix window was opened to observe the combinations of possible overlays of the two images. The gels to be matched were selected and the matching started. PROTEOMEWEAVER began the process of automatically matching stained protein regions of the overlaid gels with final matching of the gels often performed by visual inspection. Once the overlay matching has been completed, the images were viewed or printed for reference.

After the 2D gel pattern overlay for the 16 women had been obtained, the initial image comparisons were performed. The two images from the two breasts of each individual were visually inspected, aligned, and digitally assigned different colors. Typically, one pattern was assigned a green color and the other pattern a red color for analysis of the overlay in PDQUEST, while one pattern was assigned a blue color and the other pattern a yellow color for analysis using the PROTEOMEWEAVER software.

Once each image had been assigned a color, the two images to be compared were then superimposed one over the other. If the resulting overlay produced images where the predominant color of the fluorescent patterns changed from red and green to yellow and from blue and yellow to black, there was a substantially complete alignment of the proteins. When the color primarily changed to the additive color, there was no significant difference in protein expression patterns between the contralateral breasts tested. However, if there was no predominant change to the additive color, the two imaged samples expressed significantly different protein expression patterns from each other.

FIG. 1 shows the application of the method of the present invention to the nipple aspirate fluid samples from both breasts of a woman without breast cancer (normal woman). In panel A, the 2D gel pattern of the left breast was stained as red, while in panel B the pattern of the right breast was stained in green. Panel C of FIG. 1 shows the result of the overlay procedure of the method of the instant invention. The overlay is predominantly yellow because the patterns of proteins expressed are essentially the same. In all four normal individuals tested, there was a pronounced lack of differential expression in the contralateral breasts. Furthermore, there was a pronounced similarity in the protein expression patterns of the NAF samples of all four of the normal individuals.

FIG. 2 shows the application of the method of the present invention to nipple aspirate fluid samples from both breasts of a woman diagnosed with unilateral breast cancer. In panel A, the 2D gel pattern of the left breast was stained red, while in panel B the pattern of the right breast was stained green. Panel C of FIG. 2 shows that when the overlay method is used only a small portion of the proteins visualized appear as yellow. Instead most of the proteins seen in panel C of FIG. 2 appear as either red or green, indicating that the proteins in each nipple aspirate fluid sample are uniquely expressed in terms of either type of protein or level of protein (up- or down-regulated proteins). The patterns seen in FIG. 2 were typical of the patterns seen in all 12 breast cancer patients. Similar results were seen in all 12 of the patients tested. Not only do the protein expression patterns of the cancerous breasts differ from normal breasts, but also the protein expression patterns of the cancerous breasts differ from each other and from the noncancerous contralateral breast of the patient.

In addition, to the four normal women and twelve women diagnosed with unilateral breast cancer, two at-risk women having a strong familial breast cancer history, but with no evidence of breast disease by mammography or manual breast examination, were investigated. In each of the two women, the protein expression pattern of one breast resembled the normal pattern and the protein expression pattern of the other breast resembled that of a cancerous breast. The gel image overlay of the two women's right and left breasts were not predominantly the additive color and thus one of their breasts was designated as at risk for breast cancer.

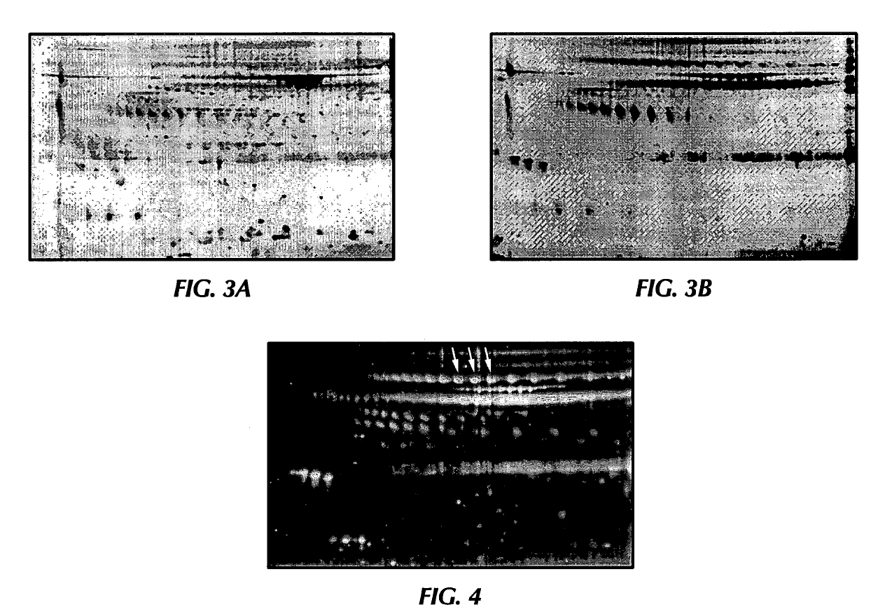

The protein expression pattern of the right and left breast of the woman having the most profound family history of breast cancer between the two at-risk women is shown in FIG. 3A-B. This woman's mother, grandmother and three maternal great aunts had breast cancer that was diagnosed when these relatives were the same age as the woman was when the NAF sample was collected. The protein expression pattern of this woman's left breast resembled the normal pattern (see FIG. 3B), while the protein expression pattern of her right breast resembled that of a cancerous breast (see FIG. 3A).

To further validate the overlay process as an early indicator of breast cancer or the risk of developing breast cancer, the NAF samples shown in FIGS. 3A and 3B were analyzed for the presence of known breast cancer markers such as HER2/neu. A number of known breast cancer markers were found in the left breast (see FIG. 3A) and not in the right breast (FIG. 3B) as shown in Table 2.

TABLE II
Known Breast Cancer Markers Found in the
Left Breast but not in the Right Breast

| Protein Marker | Mass (kDa)/pI | Mr/pI on gel |
|---|---|---|
| 14-3-3 alpha/beta | 27.7/4.79 | 27.7/4.9 |
| 14-3-3 sigma | 27.7/4.65 | 27.3/4.8 |
| 14-3-3 zeta/delta | 27.7/4.78 | 27.5/4.3 |
| Annexin I | 38.8/6.6 | 38.5/6.5 |
| Annexin III | 33.6/5.63 | 36.6/5.9 |
| Annexin V | 31/4.94 | 32.2/4.8 |
| Calreticulin | 55/4.3 | 57.8/4.6 |
| Cathepsin D | 43.2/5.9 | 41.9/5.9 |
| Cytokeratin-K8 | 53.5/5.52 | 52.0/5.5 |
| Cytokeratin K18 | 44.4/5.3 | 46.9/5.3 |
| GST | 24.2/5.6 | 24.6/5.3 |
| HER2/neu | 21.3/6.9 | 23.9/6.8 |
| HSP-27 | 27.1/5.8-6.6 | 27.9/6.5 |
| Maspin | 42.1/5.72 | 41.9/5.9 |
| PCNA | 32/4.57 | 34.1/4.7 |
| PTEN | 47.2/5.9 | 46.9/5.9 |
| Rho GDI | 27.6/4.9 | 25.1/5.0 |

Since the right breast exhibited the protein expression pattern of a normal sample and the left breast exhibited the protein expression pattern of a cancerous breast, the finding of the known breast cancer markers in the left breast and not in the right breast was a further validation of the overlay process for the detection of breast cancer. These results support the use of 2D gel electrophoresis and the overlay process of the present invention as an early indicator for breast cancer or for the risk of developing breast cancer.

Thus, visual inspection of the overlay of stained images of NAF samples from contralateral breasts of a woman or of a normal breast over another NAF sample could readily detect when one of the breasts was cancerous or at high risk of developing breast cancer. Thus, any women whose breast indicates a high risk of cancer by the overlay process described herein should become the object of increased medical surveillance at the very least.

### EXAMPLE 2

### The Identification of Acetyl-LDL Receptor as a Biomarker for Breast Cancer

FIG. 4 identifies the three major protein spots seen in all of the control NAF samples that were reduced in ten of the twelve unilateral breast cancer patients. These protein spots were excised, in-gel digested with trypsin, subjected to mass fingerprinting analysis by matrix-assisted laser desorption ionization-time of flight mass spectrometry (MALDI-TOF MS) and expert database searching.

Following differential expression analysis, these three major protein spots were carefully excised from the gel for identification. Excised gel spots were destained by washing the gel spots twice in 100 mM NH₄HCO₃ buffer, followed by soaking the gel spots in 100% acetonitrile for 10 minutes. The acetonitrile was aspirated and a trypsin solution added to the gel spots.

A small volume of a trypsin solution (approximately 5-15 μg/ml trypsin) was added to the destained gel spots and incubated for 3 hours at 37° C. or overnight at 30° C. The digested peptides were extracted, washed, desalted and concentrated before spotting the peptide samples onto the MALDI-TOF MS target.

Mass spectral analyses of the digested peptides were performed to identify the protein in the gel spots. Those of skill in the art are familiar with mass spectral analysis of digested peptides. The mass spectral analysis was conducted on a MALDI-TOF Voyager DE STR (Applied Biosystems). Spectra were carefully scrutinized for acceptable signal-to-noise ratio (S/N) to eliminate spurious artifact peaks from the peptide molecular weight lists. Both internal and external standards were employed to calibrate any shift in mass values during mass spectroscopic analysis.

Corrected molecular weight lists were then subjected to the NCBI and SwissProt databases. The NCBI database search results were displayed according the MOWSE score (a measure of the match probability between the search entry and any proteins identified from the search results). The search results also provided the number of the 94 peptides submitted that were matched and percentage of those peptides matched.

The top two matches identified by the NCBI database search were listed as human endothelial cell scavenger receptor precursor (acetyl-LDL receptor) and the human KIAA0149 gene product related to Notch 3. Not only was the MOWSE Score for each of these proteins identical (1.85×10³¹), but also both proteins matched all 94 peptides submitted with a 100% match probability. Furthermore, when the sequence alignment of the human acetyl-LDL receptor was compared with the human Notch 3 related protein using the BLOSUM-62 comparison matrix, a 99.9% identity of 830 residues of the two proteins was obtained with a gap frequency of 0.0%. Thus, the best two protein matches identified by the NCBI database (i.e., the acetyl-LDL receptor and the human KIAA0149 gene product related to Notch 3) were assumed to be the same protein, hereinafter referred to simply as the acetyl-LDL receptor. In addition, the Swiss Protein database search identified the same protein as the NCBI database (i.e., the acetyl-LDL receptor) as the closest match for the protein in the gel spots marked in FIG. 4.

Further evidence as to the significance of the identification of the protein in the gel spots as the acetyl-LDL receptor is demonstrated in that the third best match identified by the NCBI database was a human unnamed protein with a MOWSE Score of 5.52×10⁵ (as compared to 1.85×10³¹ for the acetyl-LDL receptor) and 30 of the 94 peptides matching with a 31% match probability (as compared to a 99.9% match probability for the acetyl-LDL receptor). Thus, the identification of the protein as the acetyl-LDL receptor was verified using the analytical tools of proteomic bioinformatics.

### EXAMPLE 3

### The Acetyl-LDL Receptor in Normal and Diseased Breast

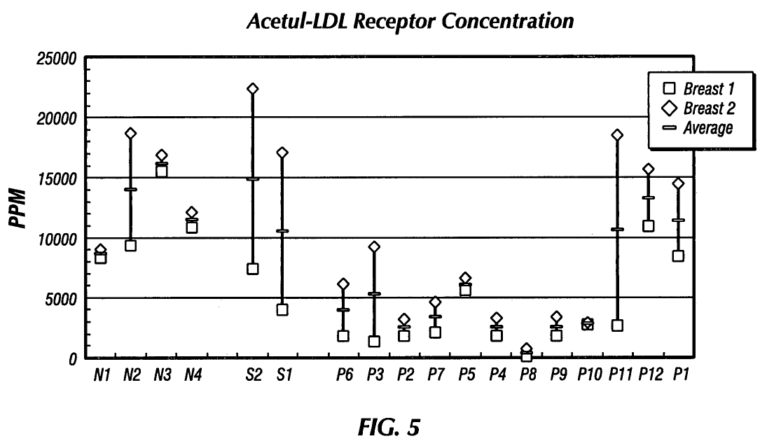

Levels of the acetyl LDL receptor were elevated in normal breasts and down-regulated in the nipple aspirate fluid sample from the cancerous breast (see FIG. 5). NAF samples were collected from both breasts of four normal women and tested for acetyl-LDL receptor concentration. The acetyl-LDL receptor concentration in the normal breasts ranged from about 8,279 ppm to about 18,669 ppm with a 95% lower confidence limit of 6,073 ppm. The mean concentration of acetyl-LDL receptor in the eight control breasts was 12,581 ppm with a standard deviation of 3,956 ppm. Thus, a normal value of acetyl-LDL receptor protein in control NAF samples was determined to be equal to or more than 8,625 ppm (the mean value minus one standard deviation) or more than or equal to 6,073 ppm (the 95% lower confidence limit of the concentration of the acetyl-LDL receptor in control breasts).

NAF samples of both breasts of the twelve unilateral breast cancer patients were also analyzed for their acetyl-LDL receptor levels. The cancerous breast of the twelve patients had an average acetyl-LDL receptor level of 3,400 ppm with a standard deviation of 3,204 ppm. Ten of the twelve patients had an acetyl-LDL receptor level in their cancerous breasts that was less than the lower 95% confidence level of the control breasts (i.e., an 83.3% correlation). FIG. 5 shows the values for each of the breasts of all twelve patients diagnosed with unilateral breast cancer (shown as P1 to P12 in FIG. 5).

In addition, to the four normal women and twelve women diagnosed with unilateral breast cancer, two at-risk women having a strong familial breast cancer history, but with no evidence of breast disease by mammography or manual breast examination, were investigated for their NAF acetyl-LDL receptor levels. As shown in FIG. 5, there was a large difference in the NAF acetyl-LDL receptor levels between the right and left breasts of these two women (S1 and S2 shown in FIG. 5). The woman, identified in FIG. 5 as S1, had the most profound family history of breast cancer between the two at-risk women. This woman's (S1) mother, grandmother and three maternal great aunts had breast cancer that was diagnosed when these relatives were the same age and the woman was when the NAF sample was collected. The acetyl-LDL receptor level in this woman's (S1) left breast was 3,920 ppm (well below the 95% confidence limit of normals), although her right breast had 17,101 ppm (well above the 95% confidence limit of normals).

These data demonstrated the validity of the method of the present invention for use in identifying protein expression patterns that are characteristic of disease states in tissues from patients. Once a differential expression pattern or disease protein footprint is identified with the method of the present invention, the differentially expressed proteins can be further explored through use of techniques to isolate and identify the proteins detected.

### EXAMPLE 4

### Differential Protein Expression Patterns in Nuerodegenerative Disease

Serum samples were collected from 22 normal or control subjects that were negative for neurodegenerative disease and from 22 patients diagnosed with amylotrophic lateral sclerosis (ALS). Using the methodology discussed above, a composite disease protein footprint for ALS was compiled.

Serum samples were aliquoted and frozen in liquid nitrogen. When the samples were thawed for analysis, a protein fraction was precipitated, washed and then subjected to 2D gel electrophoresis as described above.

The 2D gel patterns were stained with SYPRO RUBY fluorescent stain (Bio-Rad Laboratories), destained, and scanned. The 2D gel scans were digitized for further analysis. The 22 normal gel patterns were compared and a composite gel pattern representing the normal protein expression pattern or normal protein footprint was derived.

This normal protein expression pattern was then compared to the gel pattern obtained in the 22 ALS patients. Eight proteins were routinely found to be differentially expressed between the ALS patients and the normal subjects. These 8 proteins make up the disease protein footprint. Furthermore, patients diagnosed with Parkinson's disease and Alzheimer's disease were also found to have many of the same 8 proteins differentially expressed when compared to normal subjects.

### EXAMPLE 5

### Protein 4411 in Normal Subjects and Patients with Nuerodegenerative Disease

One of the differentially expressed protein spots (protein 4411) was carefully excised from the gel, digested with trypsin, subjected to mass fingerprinting analysis by MALDI-TOF MS, and identified by expert database searching of the results as described above. Protein 4411 was found to contain a number of peptides that matched peptide sequences in the acetyl-LDL receptor. Thus, protein 4411 was identified as being related to the acetyl-LDL receptor.

Protein 4411 concentration was determined in 24 normal subjects, 92 ALS patients, 36 Alzheimer patients, and 26 Parkinson patients. Normal serum levels of protein 4411 ranged from an undetectable 0 ppm to about 320 ppm, with a mean value of 32.6 ppm±43.7 S.E. The concentration of protein 4411 in the nuerodegenerative patients was as follows: the mean concentration of protein 4411 in the 92 ALS patients was 245.3±22.3 S.E. ppm; the mean concentration of protein 4411 in the 36 Alzheimer patients was 394.3±35.6 S.E. ppm; and the mean concentration of protein 4411 in the 26 Parkinson patients was 625.1±41.9 S.E. ppm as shown in Table 3.

TABLE 3

| Diagnosis | # of Patients | Mean Value | Standard Error |
|---|---|---|---|
| Normal | 24 | 32.6 | 43.7 |
| ALS | 92 | 245.3 | 22.3 |
| Alzheimer | 36 | 394.3 | 35.6 |
| Parkinson | 26 | 625.1 | 41.9 |

The test results were subjected to a Bonferroni (pairwise) multiple comparison analysis. The Bonferroni analysis found that normal subjects were significantly differentiated from Alzheimer and Parkinson patients and that ALS patients were significantly differentiated from Parkinson patients based on the level of protein 4411 in a serum sample. However, final differentiation of ALS patients from normal subjects and Alzheimer from Parkinson patients requires additional testing.

The methods disclosed herein can also be applied to analysis of diseases not disclosed or altered biological states not disclosed, including but not limited to other neurodegenerative diseases, other forms of cancer, autoimmune diseases, immune system dysfunction, drug resistance or drug allergy or sensitivity. While the methods have been described in terms of preferred embodiments, it will be apparent to those of skill in the art that variations may be applied to the methods including the sequence of steps in the methods. Certain agents may be substituted by one of skill and similar results may be achieved, as will be appreciated by one of skill in the art. Such modifications or substitutions to the methods of the present invention are deemed to be within the spirit, scope and concept of the invention as defined by the disclosure and its claims.

What is claimed is:

1. A method for determining an altered pattern of protein expression comprising:
a) collecting a biological sample from an individual having an altered biological state;
b) performing a two-dimensional (2D) electrophoretic separation of a plurality of proteins in the biological sample to produce a sample 2D gel pattern;
c) coloring the sample 2D gel pattern a first color;
d) superimposing the sample 2D gel pattern over a control 2D gel pattern colored a second color, wherein the control 2D gel pattern represents a standard protein expression pattern of a control sample collected from an individual free of the altered biological state;
e) aligning a set of standard proteins in the sample 2D gel pattern and the control 2D gel pattern to form an aligned overlay; and
f) conducting an image analysis of the aligned overlay to identify and quantify a set of protein variations in the sample 2D gel pattern that differ from the control 2D gel pattern, whereby the set of protein variations is indicative of the altered biological state.

2. The method of claim 1, wherein the altered biological state is a cancerous condition.

3. The method of claim 1, wherein the altered biological state is breast cancer.

4. The method of claim 1, wherein the altered biological state is a nuerodegenerative disease.

5. The method of claim 1, wherein the altered biological state is ALS, Parkinson's disease, or Alzheimer's disease.

6. The method of claim 1, further comprising separating a protein fraction from the biological sample prior to performing the 2D electrophoretic separation of the proteins.

7. The method of claim 6, wherein the protein fraction is separated by precipitation with ammonium sulfate, trichloroacetic acid, perchloric acid, acetone, ethanol, or a combination of the same.

8. The method of claim 1, wherein the sample 2D gel pattern and the control 2D gel pattern are colored with visible stains.

9. The method of claim 1, further comprising staining the sample 2D gel pattern.

10. The method of claim 9, wherein the sample 2D gel pattern is stained with a fluorescent stain.

11. The method of claim 9, further comprising digitalizing an image of the stained sample 2D gel pattern.

12. The method of claim 11, wherein the digitalized image is colored the first color.

13. The method of claim 1, wherein a digitalized image of the sample 2D gel pattern is electronically colored the first color and a digitized image of the control 2D gel pattern is electronically colored the second color.

14. The method of claim 1, wherein the first color and the second color are at opposed ends of the color spectra.

15. The method of claim 1, wherein the two-dimensional electrophoretic separation comprises a separation by isoelectric point followed by a separation by molecular weight.

16. The method of claim 1, further comprising the steps of:
g) performing steps a)-f) on a number of biological samples from individuals having the altered biological state; and
h) preparing a composite gel pattern of the set of protein variations found in the biological samples.

17. A method for screening for an altered biological state comprising:
a) collecting a biological sample from a subject;
b) performing a two-dimensional (2D) electrophoretic separation of a plurality of proteins in the biological sample to produce a sample 2D gel pattern;
c) coloring the sample 2D gel pattern a first color;
d) superimposing the sample 2D gel pattern over a control 2D gel pattern colored a second color, wherein the control 2D gel pattern represents a standard protein expression pattern of a control sample collected from an individual free of the altered biological state;
e) aligning a standard protein in the sample 2D gel pattern with the standard protein in the control 2D gel pattern to form an aligned overlay; and
f) conducting an image analysis of the overlay to identify and quantify a set of protein variations in the sample 2D gel pattern that differ from the control 2D gel pattern, whereby the set of protein variations is indicative of the altered biological state.

18. The method of claim 17, wherein the altered biological state is a cancerous condition.

19. The method of claim 17, wherein the altered biological state is breast cancer.

20. The method of claim 17, wherein the altered biological state is a nuerodegenerative disease.

21. The method of claim 17, wherein the altered biological state is ALS, Parkinson's disease, or Alzheimer's disease.

22. The method of claim 17, further comprising separating a protein fraction from the biological sample prior to performing the 2D electrophoretic separation of the proteins.

23. The method of claim 22, wherein the protein fraction is separated by precipitation with ammonium sulfate, trichloroacetic acid, perchloric acid, acetone, ethanol, or a combination of the same.

24. The method of claim 17, wherein the sample 2D gel pattern and the control 2D gel pattern are colored with visible stains.

25. The method of claim 17, further comprising staining the sample 2D gel pattern.

26. The method of claim 25, wherein the sample 2D gel pattern is stained with a fluorescent stain.

27. The method of claim 25, further comprising digitalizing an image of the stained sample 2D gel pattern.

28. The method of claim 27, wherein the digitalized image is colored the first color.

29. The method of claim 25, wherein a digitalized image of the sample 2D gel pattern and the control 2D gel pattern are electronically colored the first color and the second color.

30. The method of claim 17, wherein the first color and the second color are at opposed ends of the color spectra.

31. The method of claim 17, wherein the two-dimensional electrophoretic separation comprises a separation by isoelectric point followed by a separation by molecular weight.

32. A method of using protein expression patterns to diagnose an altered biological state comprising:
a) collecting a biological sample from a patient;
b) performing a two dimensional (2D) electrophoretic separation of a plurality of proteins in the biological sample to produce a sample 2D gel pattern;
c) superimposing the sample 2D gel pattern over a composite gel pattern of protein expression determined according to claim 16 to form a gel pattern overlay; and
d) conducting an image analysis of the overlay to diagnose the patient.

33. The method of claim 32, wherein the altered biological state is a cancerous condition.

34. The method of claim 32, wherein the altered biological state is breast cancer.

35. The method of claim 32, wherein the altered biological state is a nuerodegenerative disease.

36. The method of claim 32, wherein the altered biological state is ALS, Parkinson's disease, or Alzheimer's disease.

37. A method for determining a pattern of protein expression for an altered biological state comprising:
a) collecting a first biological sample known to exhibit the altered biological state;
b) collecting a second biological sample known not to exhibit the altered biological state;
c) precipitating a first protein fraction from the first sample and a second protein fraction from the second sample;
d) performing a two-dimensional gel electrophoretic analysis of the first protein fraction to produce a first 2D gel pattern;
e) performing a two-dimensional gel electrophoretic analysis of the second protein fraction to produce a second 2D gel pattern;
f) staining the first 2D gel pattern a first color and the second 2D gel pattern a second color;
g) superimposing the first 2D gel pattern over the second 2D gel pattern to form an overlay;
h) aligning the first 2D gel pattern with the second 2D gel pattern to maximize the presence of a third color in the overlay, wherein the third color results from mixing the first color and the second color;
i) conducting an image analysis of the aligned overlay to identify a set of differentially expressed proteins in the first sample, whereby the set of differentially expressed proteins is indicative of the altered biological state.

38. The method of claim 37, further comprising the step of using the set of differentially expressed proteins to diagnose the altered biological state.

39. The method of claim 37, wherein the first and second colors are at opposed ends of the color spectra.

40. The method of claim 37, wherein the first and second protein fractions are precipitated with ammonium sulfate, trichloroacetic acid, perchloric acid, acetone, ethanol, or a combination of the same.

41. A method for screening for breast cancer comprising:
a) collecting a ductal fluid sample from a breast of a subject;
b) precipitating a protein fraction of the ductal fluid sample;
c) performing a two dimensional (2D) electrophoretic separation of a plurality of proteins in the protein precipitate to produce a sample 2D gel pattern;
d) staining the sample 2D gel pattern;
e) digitizing an image of the stained sample 2D gel pattern and assigning the digitalized image a first color;
d) superimposing the sample 2D gel pattern over a digitized image of a control 2D gel pattern, wherein the control 2D gel pattern represents a standard protein expression pattern of a noncancerous breast ductal fluid assigned a second color;
e) aligning the sample 2D gel pattern and the control 2D gel pattern to form an overlay;
f) conducting an image analysis of the overlay; and
g) using the image analysis of the overlay as a risk indicator of the breast for breast cancer.
